(19) United States
(12) Patent Application Publication
(10) Pub. No.: US 2026/0204431 A1
(43) Pub. Date: Jul. 16, 2026

(54) INTERVIEW-RECORD-ENTERING SUPPORT APPARATUS, INTERVIEW-RECORD-ENTERING SUPPORT METHOD AND PROGRAM

(71) Applicant: NTT,Inc., Tokyo (JP)

(72) Inventors: Tae SATO, Tokyo (JP); Kaori FUJIMURA, Tokyo (JP); Taiga SANO, Tokyo (JP); Yasuo ISHIGURE, Tokyo (JP); Asami MIYAJIMA, Tokyo (JP)

(21) Appl. No.: 19/133,417

(22) PCT Filed: Dec. 5, 2022
(86) PCT No.: PCT/JP2022/044742

§ 371 Date: May 28, 2025

(51) Int. Cl. G16H 80/00

(52) U.S. Cl. CPC G16H 80/00

(57) ABSTRACT

An interview record input support apparatus includes a selection unit configured to accept selection of one or more elements performed in a certain interview from among a plurality of the elements configuring a process of conversation between an interviewer and an interviewee, and a registration unit configured to accept a record regarding the certain interview for an item associated with any of the selected elements from among a plurality of items that are record target candidates for the interview in order to review the interview, and register the record in a storage unit, thereby improving efficiency of inputting the record regarding the interview.

### TECHNICAL FIELD

The present invention relates to an interview record input support apparatus, an interview record input support method, and a program.

### BACKGROUND ART

It is important to support behavioral changes in which a person who should improve his/her behavior such as to prevent a lifestyle-related disease takes action by himself/herself.

Therefore, in specific health guidance, an interview for promoting a behavioral change by a professional (a public health nurse, an administrative dietitian, or the like) is performed.

In recording of a health guidance interview, there is a system in which an interviewer reviews the interview by registering health problems and behavior plan content possessed by an interviewee (a person who receives assistance with a behavioral change).

### Citation List

### Non Patent Literature

Non Patent Literature 1: Kana KAZAWA, Naoko TAKEYAMA, Madoka KAWAI, Keiko UMEKI, Tokika MAZAKI, Ayako TSUNOI, Hiroko HOSOKAWA, Michiko MORIYAMA, Syunichi KURINO, and Noriaki YOSHIKAI, “Intermediate report of development of health instruction inquiry/intervention support system for supporting behavioral change and self-management ability of chronic disease patients”, Journal of the Medical Information Society of Japan (2187-1469), volume 20, Page 103-106 (2019.09)

### SUMMARY OF INVENTION

### Technical Problem

In the conventional system, it is necessary to input all items that are recording target candidates in an interview one by one, and it takes time and effort to create a record regarding the interview.

The present invention has been made in view of the above points, and an object thereof is to improve efficiency of inputting a record regarding an interview.

### Solution to Problem

Therefore, in order to solve the above problems, an interview record input support apparatus includes a selection unit configured to accept selection of one or more elements performed in a certain interview from among a plurality of the elements configuring a process of conversation between an interviewer and an interviewee; and a registration unit configured to accept a record regarding the certain interview for an item associated with any of the selected elements from among a plurality of items that are record target candidates for the interview in order to review the interview, and register the record in a storage unit.

### Advantageous Effects of Invention

It is possible to improve efficiency of inputting a record regarding an interview.

### BRIEF DESCRIPTION OF DRAWINGS

FIG. 1 is a diagram illustrating a hardware configuration example of an interview record input support apparatus 10 according to an embodiment of the present invention.

FIG. 2 is a diagram illustrating a functional configuration example of the interview record input support apparatus 10 according to the embodiment of the present invention.

FIG. 3 is a flowchart for describing an example of a processing procedure executed by an executed conversation process management unit 11.

FIG. 4 is a diagram illustrating a configuration example of a common conversation process model DB 121.

FIG. 5 is a diagram illustrating a display example in a case where a conversation process element at Lv.1 is set as a display target.

FIG. 6 is a diagram illustrating a display example in a case where a conversation process element at Lv.2 is set as a display target.

FIG. 7 is a diagram illustrating a configuration example of a conversation process execution recording DB 122.

FIG. 8 is a flowchart for describing an example of a processing procedure executed by an interview content registration unit 12.

FIG. 9 is a diagram illustrating a configuration example of an acquired data item ID attribute table 123-1 configuring an interview manual recording item DB 123.

FIG. 10 is a diagram illustrating an example of a tree structure 123-2 configuring the interview manual recording item DB 123.

FIG. 11 is a diagram illustrating a first display example of an individual interview record input screen.

FIG. 12 is a diagram illustrating a second display example of the individual interview record input screen.

FIG. 13 is a diagram illustrating a configuration example of an individual interview manual recording DB 124.

FIG. 14 is a flowchart for describing an example of a processing procedure executed by a reason input candidate management unit 13.

FIG. 15 is a diagram illustrating a configuration example of a reason record information recording table 125-1.

FIG. 16 is a diagram illustrating a calculation example of a reason candidate score of a parent.

FIG. 17 is a diagram illustrating a configuration example of a reason reference destination candidate ID management table 125-2.

FIG. 18 is a diagram illustrating a display example of a visualization screen 550.

FIG. 19 is a flowchart for describing an example of a processing procedure executed by an interview case visualization unit 14.

FIG. 20 is a diagram illustrating a configuration example of a visualization definition DB 126.

### DESCRIPTION OF EMBODIMENTS

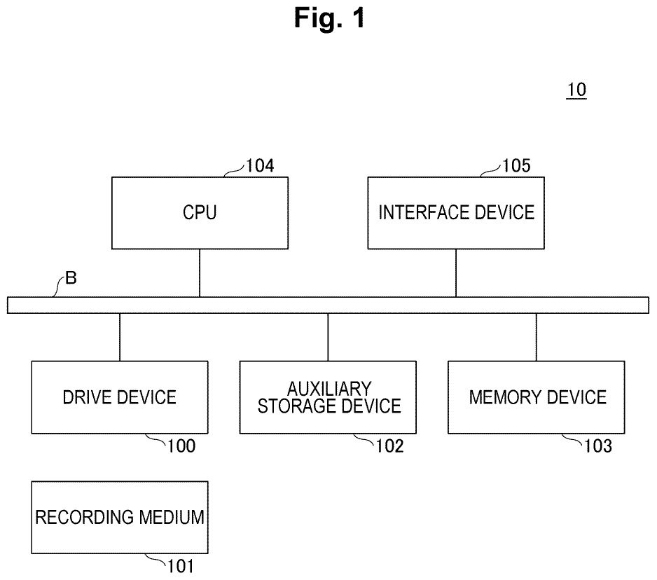

Hereinafter, embodiments of the present invention will be described with reference to the drawings. FIG. 1 is a diagram illustrating a hardware configuration example of an interview record input support apparatus 10 according to an embodiment of the present invention. The interview record input support apparatus 10 in FIG. 1 includes a drive device 100, an auxiliary storage device 102, a memory device 103, a CPU 104, an interface device 105, a display device 106, an input device 107, and the like which are connected to each other via a bus B.

A program for realizing processing in the interview record input support apparatus 10 is provided by a recording medium 101 such as a CD-ROM. When the recording medium 101 storing the program is set in the drive device 100, the program is installed in the auxiliary storage device 102 from the recording medium 101 via the drive device 100. However, the program is not necessarily installed from the recording medium 101, and may be downloaded from another computer via a network. The auxiliary storage device 102 stores the installed program and also stores required files, data, and the like.

In a case where an instruction to start the program is given, the memory device 103 reads the program from the auxiliary storage device 102 and stores the program. The CPU 104 realizes a function related to the interview record input support apparatus 10 according to the program stored in the memory device 103. The interface device 105 is used as an interface for connection to a network. The display device 106 displays a graphical user interface (GUI) or the like by the program. The input device 107 includes a keyboard, a mouse, and the like, and is used to input various operation commands.

Note that the interview record input support apparatus 10 does not need to include the display device 106 and the input device 107. In this case, for example, a terminal connected to the interview record input support apparatus 10 via a network may function as the display device 106 and the input device 107. Examples of such a terminal include a personal computer (PC), a smartphone, a tablet terminal, and the like.

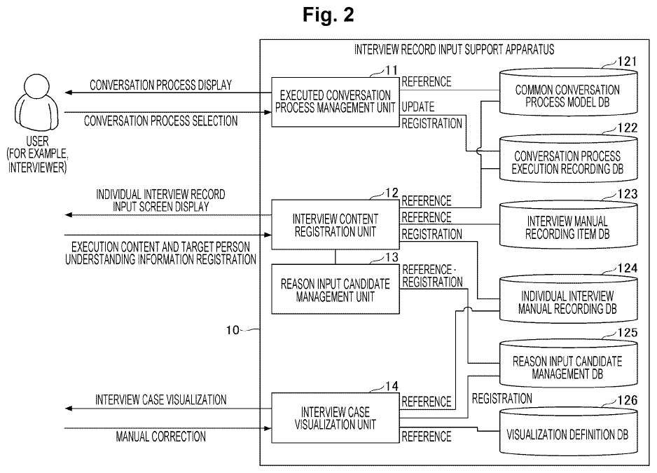

FIG. 2 is a diagram illustrating a functional configuration example of the interview record input support apparatus 10 according to the embodiment of the present invention. In FIG. 2, the interview record input support apparatus 10 includes an executed conversation process management unit 11, an interview content registration unit 12, a reason input candidate management unit 13, and an interview case visualization unit 14. Each of these units is realized by one or more programs installed in the interview record input support apparatus 10 causing the CPU 104 to execute processing. The interview record input support apparatus 10 also uses databases (storage units) such as a common conversation process model DB 121, a conversation process execution recording DB 122, an interview manual recording item DB 123, an individual interview manual recording DB 124, a reason input candidate management DB 125, and a visualization definition DB 126. Each of these databases can be realized by using, for example, the auxiliary storage device 102 or a storage device connectable to the interview record input support apparatus 10 via a network.

Hereinafter, a processing procedure executed by the interview record input support apparatus 10 will be described.

### Executed Conversation Process Management Unit 11

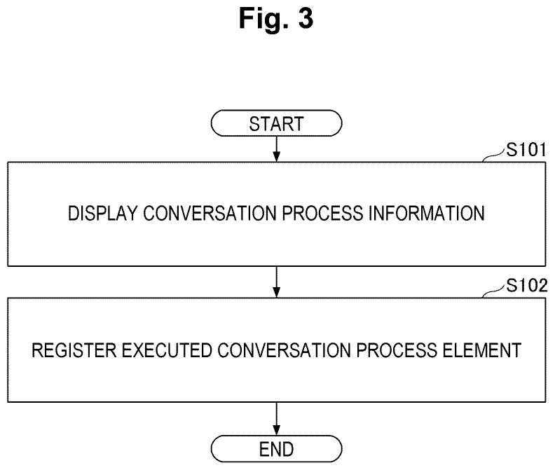

FIG. 3 is a flowchart for describing an example of a processing procedure executed by the executed conversation process management unit 11. The processing procedure in FIG. 3 is executed in order for a certain interviewer (a public health nurse or the like) to execute a certain interview (hereinafter referred to as a “target interview”) with an interviewee (hereinafter referred to as a “target person”) or the like and then register information regarding the target interview. Note that, as an example of a target person, there is a person who receives assistance with a behavioral change.

In step S101, the executed conversation process management unit 11 displays a screen indicating conversation process information registered in the common conversation process model DB 121 on the display device 106 in response to a conversation process display request from a user (interviewer).

In the common conversation process model DB 121, information (conversation process information) regarding an element configuring a conversation or a process (hereinafter referred to as a “conversation process”) between an interviewer (a public health nurse or the like) and an interview target person (hereinafter simply referred to as a “target person”) or the like in one interview is registered in advance. That is, the conversation process information stored in the common conversation process model DB 121 is not a DB that stores information regarding a specific interview individually performed, but is information regarding a typical or general configuration of a conversation process in an interview.

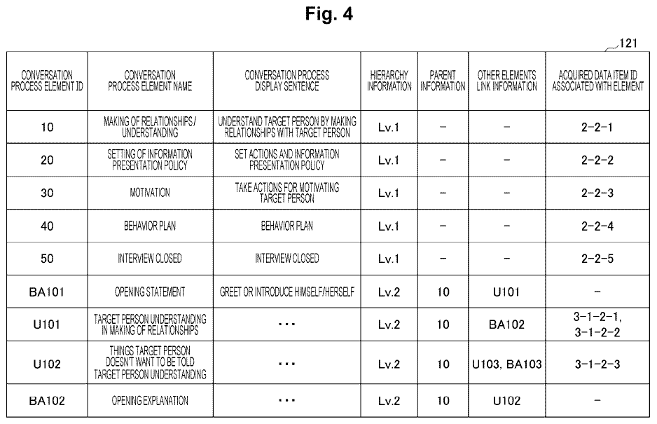

FIG. 4 is a diagram illustrating a configuration example of the common conversation process model DB 121. As illustrated in FIG. 4, the common conversation process model DB 121 stores in advance, for each element that may configure a conversation process (steps or units for dividing a conversation process in one interview according to a role thereof; hereinafter referred to as a “conversation process element”), an element ID, an element name, a display sentence, hierarchical information, parent information, other element link information, an acquired data item ID associated with an element, and the like.

The element ID is identification information of a conversation process element. The element name is a name given to the conversation process element. The element display sentence is a text for display representing the conversation process element. The hierarchical information is information expressing a hierarchy (level) in a hierarchical relationship (parent-child relationship) between the conversation process elements in a format of “Lv. N”. Here, N is a numerical value indicating the hierarchy from the uppermost in the hierarchical relationship (parent-child relationship). The parent information is an element ID of a conversation process element serving as a parent in a hierarchical relationship between conversation process elements. The other element link information is information indicating an element ID of a conversation process element in the next order in a temporal order relationship (context) between the conversation process elements. In a case where the order relationship of the conversation process elements based on the other element link information at Lv. N extends over a plurality of conversation process elements at Lv. N-1 (that is, a parent hierarchy), there is a temporal context between the plurality of conversation process elements at Lv. N-1. The context of a conversation process element of which the hierarchical information is “Lv. 1”, which does not have such a context, follows the record order of the common conversation process model DB 121. The acquired data item ID associated with the element is an acquired data item ID associated with (related to) the conversation process element. The meaning of the acquired data item ID will be described later.

The executed conversation process management unit 11 may restrict conversation process information that is a display target at the beginning on the basis of the hierarchy information. By doing so, it is possible to avoid complication of the content of the screen to be displayed.

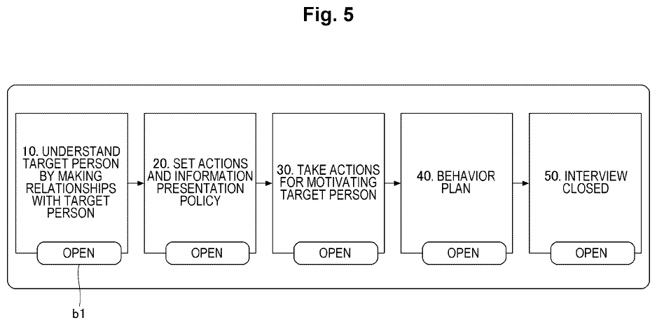

FIG. 5 is a diagram illustrating a display example in a case where the conversation process element at Lv. 1 is a display target. In FIG. 5, each rectangle corresponds to a conversation process element at Lv. 1, and includes a “conversation process display sentence” stored in the common conversation process model DB 121 (FIG. 4) for the corresponding conversation process element. The arrows connecting the rectangles indicate a temporal order relationship between the conversation process elements.

Each rectangle includes a button for expanding each conversation process element. For example, when a button b1 corresponding to a conversation process element of which an element ID is “10” is pressed, the executed conversation process management unit 11 displays a child element (conversation process element at Lv. 2) of the conversation process element.

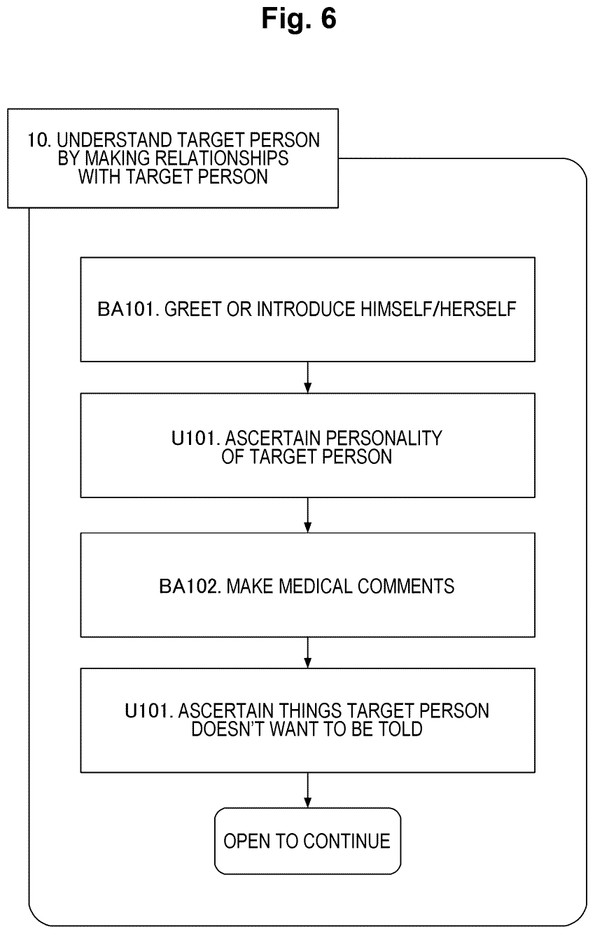

FIG. 6 is a diagram illustrating a display example in a case where the conversation process element at LV. 2 is set as a display target. FIG. 6 illustrates a context between child elements of the conversation process element of which an element ID is “10”.

The user selects a conversation process element executed in a target interview among conversation process elements displayed on the screen. In this case, the conversation process elements are selected in the order of execution. Alternatively, selection of the executed conversation process elements may not be performed in the order of execution. In this case, the executed conversation process management unit 11 may display a list of the selected conversation process elements (that is, a list of conversation process elements executed by a user), and the selected conversation process elements may be arranged in the execution order through an operation such as drag-and-drop on the list.

That is, in the common conversation process model DB 121, the conversation process elements that can configure the conversation process are washed out and registered, and thus, not all the conversation process elements are executed in each interview. Thus, here, a selection of a conversation process element is received from the user in order to narrow down executed conversation process elements.

When the selection of the conversation process element is received from the user, the executed conversation process management unit 11 registers information regarding the conversation process element executed on the target interview by the user in the conversation process execution recording DB 122 on the basis of the selection result of the user (S102).

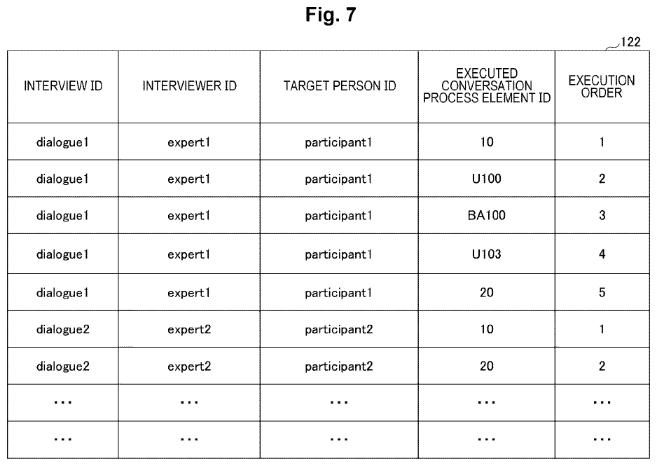

FIG. 7 is a diagram illustrating a configuration example of the conversation process execution recording DB 122. As illustrated in FIG. 7, the conversation process execution recording DB 122 stores a record including an interview ID, an interviewer ID, a target person ID, an executed conversation process ID, an execution order, and the like for each conversation process element executed in each interview performed in the past.

The interview ID is identification information of the interview. The interviewer ID is identification information of a person who performed an interview in the interview (interviewer). The target person ID is identification information of a target person of the interview. The executed conversation process element ID is a conversation process element ID of a conversation process element executed in the interview. The execution order is an order in which the conversation process element was executed in the interview.

Note that the interview ID, the interviewer ID, and the target person ID are input by the user at the start of the processing procedure in FIG. 3, for example. The executed conversation process ID and the execution order are specified by the executed conversation process management unit 11 on the basis of an operation on the screen in FIGS. 5 and 6.

### Interview Content Registration Unit 12

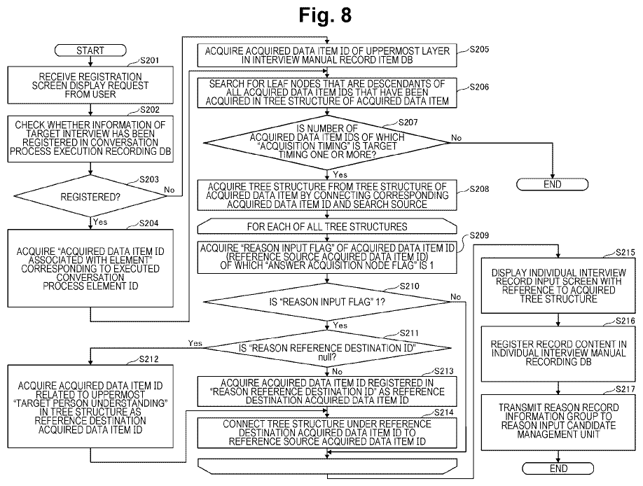

When the processing procedure in FIG. 3 is ended for the target interview, the processing procedure in FIG. 8 is subsequently executed. However, for the target interview, the processing procedure in FIG. 8 may be executed without executing the processing procedure in FIG. 3.

FIG. 8 is a flowchart for describing an example of a processing procedure executed by the interview content registration unit 12. The processing procedure in FIG. 8 is executed in order for the user (interviewer) to register interview information ((1) information regarding target person understanding, (2) information regarding intervention, and (3) information regarding a reason for intervention) for reviewing an interview in the interview record input support apparatus 10.

In step S201, the interview content registration unit 12 accepts a display request for a registration screen from the user. In this case, the user inputs an interview ID of the target interview (hereinafter referred to as a “target interview ID”) and a value (for example, “after interview”) indicating an acquisition timing (hereinafter referred to as a “target acquisition timing”). The acquisition timing will be described later.

Subsequently, the interview content registration unit 12 checks whether or not a record (that is, a record in which the target interview ID is included as the “interview ID”) of the target interview is registered in the conversation process execution recording DB 122 (S202).

In a case where the record of the target interview is registered in the conversation process execution recording DB 122 (FIG. 7) (Yes in S203), the interview content registration unit 12 acquires an “acquired data item ID associated with element” that is associated with the “executed conversation process element ID” in the common conversation process model DB 121 (FIG. 4) for each record (S204). As a result, the acquired data item IDs possibly executed in the target interview are narrowed down on the basis of the conversation process element. For example, when the target interview ID is “dialogue 1”, the “executed conversation process element ID” of each record of the target interview ID in the conversation process execution recording DB 122 (FIG. 7) is 10, U100, BA100, U103, or 20. The interview content registration unit 12 acquires the “acquired data item ID associated with element” stored in the common conversation process model DB 121 (FIG. 4) with respect to these conversation process element IDs. In this case, acquired data item IDs such as 2-2-1, . . . , 3-1-2-1, 3-1-2-2, . . . , and 2-2-2 are acquired.

On the other hand, in a case where the record of the target interview is not registered in the conversation process execution recording DB 122 (FIG. 7) (No in S203), the interview content registration unit 12 acquires all the acquired data item IDs of the uppermost layer in the interview manual recording item DB 123 (S205).

The interview manual recording item DB 123 is a database in which information for defining items (for example, “what was the blood glucose level of this target person?” and “what was the personality tendency?”) that are recording target candidates in an individual interview for review of the interviewer is registered in advance. In the present embodiment, items that are record target candidates (hereinafter referred to as “record candidate items”) are roughly divided into items related to target person understanding and items related to intervention. The target person understanding indicates that the interviewer ascertains (understands) a situation of a target person during an interview. The intervention indicates all actions (questions, comments, and the like) performed by an interviewer on a target person during an interview. For example, listening to (understanding) a target person's lifestyle and saying “Since there are a lot of carbohydrates, how about reducing the amount of rice?” corresponds to the intervention. In addition, the intervention includes greetings by an interviewer to build a good relationship with a target person during an interview. In the present embodiment, it is assumed that the reason the interviewer executed the intervention is also recorded for the intervention. For example, for the intervention of “explained the lifestyle disease”, for example, the reason “The target person A was a person who had no knowledge of health” is recorded. This is based on the premise that it is worth learning how to intervene in accordance with the characteristics of a target person in reviewing the interview.

In the interview manual recording item DB 123, not only the record candidate items but also information (for example, information indicating that the blood glucose level is one of the biological aspects) regarding a category for classifying the record candidate items in order to facilitate input of values to the record candidate items are registered.

In the present embodiment, the “record candidate item” and the “category” will be collectively referred to as an “acquired data item”, and the identification information of the acquired data item will be referred to as an “acquired data item ID”. The “record candidate item” may belong to (be classified into) one of the “categories”, and the “category” may further belong to a higher (parent) “category”. That is, the categories have a hierarchical relationship (parent-child relationship). Therefore, the acquired data item ID group forms a hierarchical relationship (parent-child relationship). The interview manual recording item DB 123 stores tree structure information (hereinafter referred to as a “tree structure 123-2”) representing this hierarchical relationship and a table (hereinafter referred to as “acquired data item ID attribute table 123-1”) in which attribute information of each acquired data item ID (a “record candidate item” or a “category”) is registered.

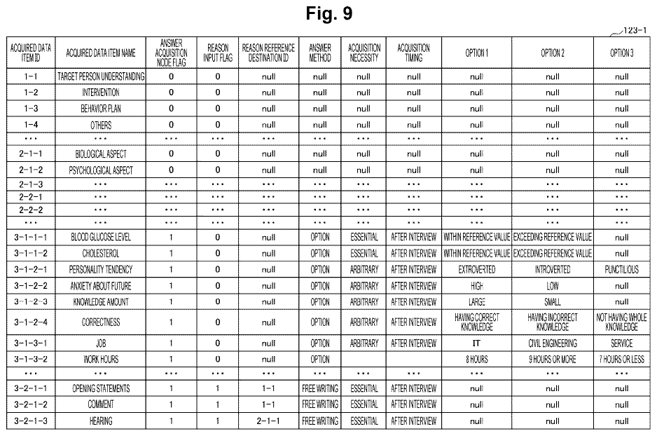

FIG. 9 is a diagram illustrating a configuration example of the acquired data item ID attribute table 123-1 configuring the interview manual recording item DB 123. As illustrated in FIG. 9, in the acquired data item ID attribute table 123-1, an acquired data item name, an answer acquisition node flag, a reason input flag, a reason reference destination ID, an answer method, acquisition necessity, an acquisition timing, options 1 to 3, and the like are registered in advance for each acquired data item ID.

The acquired data item name is the name of the acquired data item ID, and is mainly used for display. The answer acquisition node flag is an item indicating whether or not the item is an item of which a value is input by the user (that is, whether or not the item is a record candidate item). “1” indicates a record candidate item, and “0” indicates a category.

The reason input flag indicates necessity of input of a reason for a record in the acquired data item ID (why has there been performed as indicated by the record?). “1” indicates that the reason needs to be input, and “0” indicates that the reason does not need to be input. In the present embodiment, acquired data item IDs are roughly divided into IDs related to target person understanding and IDs related to intervention, and an ID in which the reason needs to be input is the acquired data item ID. This is because, in the case of an intervention, it is important for the review of the interview to record why such an intervention has been performed. Therefore, the reason input flag of the acquired data item ID related to the intervention and the record candidate item is “1”, and the reason input flag of the other acquired data item ID is “0”. The reason reference destination ID is an item in which the acquired data item ID that is a reason candidate is registered for the acquired data item ID of which the reason input flag is “1”. That is, in the present embodiment, the reason for the acquired data item ID (record candidate item) related to the intervention is selected from the records for the other acquired data item IDs. The acquired data item ID that can be a reason is an acquired data item ID related to target person understanding. This is because understanding a target person is a reason for the intervention since the intervention occurs by understanding the target person. That is, there is a causal relationship between the target person understanding and the intervention.

The acquisition necessity is an item indicating whether or not recording by the user is essential. “1” indicates that input of a value by the user is essential, and “0” indicates that it is not essential. Therefore, the acquired data item ID of which the acquisition necessity is “1” indicates a record candidate item, and the acquired data item ID of which the acquisition necessity is “0” indicates a category. This is because the category is merely for classifying the record candidate items and is not a recording target.

The acquisition timing is an item indicating a timing at which recording is performed (a timing at which the interview content registration unit 12 accepts the recording). Since the recording target is a record candidate item, the acquisition timing is also an item valid for the record candidate item. The acquired data item ID of which an acquisition timing is “after interview” indicates a record candidate item to be recorded after the end of the interview.

The options 1 to 3 are items valid for the record candidate item and indicate options (candidates) of values to be recorded for the record candidate item. The acquired data item ID that corresponds to the record candidate item and for which no option is registered indicates that the content of the record is free-form. The upper limit of the number of options may not be three.

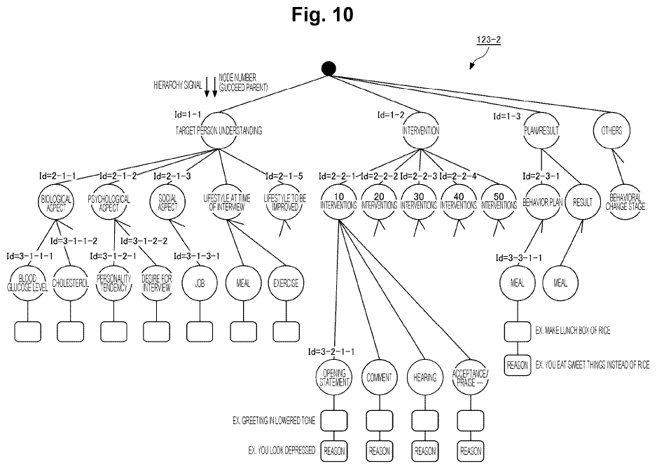

FIG. 10 is a diagram illustrating an example of the tree structure 123-2 configuring the interview manual recording item DB 123. In FIG. 10, each node represented by a circle corresponds to one acquired data item ID, and a parent-child relationship of the nodes indicates a parent-child relationship between the acquired data item IDs. A terminal node among the nodes of the circles corresponds to the record candidate item, and a joint node corresponds to the category. Hereinafter, the terminal node among the nodes of the circles will also be referred to as a “leaf node”.

A rounded rectangular node that is a child node of the leaf node is not a node corresponding to the acquired data item ID, and for convenience, represents that the “answer acquisition node flag” of the acquired data item ID corresponding to the leaf node is “1”. In addition, the rounded rectangular node including the text “reason”, which is a grandchild node of the leaf node, is not a node corresponding to the acquired data item ID, and for convenience, represents that the “reason input flag” of the acquired data item ID corresponding to the leaf node is “1”. Therefore, the rounded rectangular node is not strictly a node of a tree structure indicating a parent-child relationship between the acquired data item IDs. In the following description, a rounded rectangular node is not handled as a node of the tree structure. Therefore, the “leaf node” in the following description is a “terminal node” and is a leaf node among the nodes of the circles in FIG. 10.

In step S205 described above, all the acquired data item IDs corresponding categories of the uppermost layer in the tree structure 123-2 (FIG. 10) are acquired. In the example in FIG. 10, four acquired data item IDs are acquired. That is, in a case where the record of the target interview is not registered in the conversation process execution recording DB 122 (FIG. 7), acquired data item IDs possibly executed in the target interview cannot be narrowed down on the basis of the conversation process element, and thus all the acquired data item IDs of the uppermost layer are acquired. In the present embodiment, the acquired data item IDs are roughly divided into the target person understanding and the intervention. However, strictly speaking, since an acquired data item ID belonging to another concept (“plan/result”, “others”, and the like in FIG. 10) also exists, a category other than the target person understanding and the intervention is also illustrated in FIG. 10 as the category of the uppermost layer.

Subsequent to step S204 or S205, the interview content registration unit 12 refers to the tree structure 123-2 (FIG. 10), and searches for acquired data item IDs corresponding to all the leaf nodes that are descendants of the acquired data item ID for each acquired data item ID acquired in step S204 or S205 (S206). Only the acquired data item ID of which the acquisition necessity flag is “essential” may be set as a search target. In addition, in the interview manual recording item DB 123, a column of an answerer flag (a flag indicating who is to input an answer (a public health nurse who performed an interview, a visited senior public health nurse, or the like) ) may be provided for each acquired data item ID, and only an acquired data item ID matching an answerer flag specified at the start in FIG. 8 may be set as a search target.

Following step S206, the interview content registration unit 12 determines whether or not there are one or more acquired data item IDs of which the “acquisition timing” (FIG. 9) is a target acquisition timing (for example, “after interview”) among the acquired data item IDs related to the retrieved leaf node (S207). The “acquisition timing” is a valid item for the acquired data item ID that is a record candidate item, and is an item indicating a timing at which the interview record input support apparatus 10 acquires a record for the record candidate item (in other words, a timing at which the user inputs the record for the record candidate item to the interview record input support apparatus 10).

If the determination result is negative (No in S207), the process in FIG. 8 is ended. This is because, in this case, the user cannot input a desired acquired data item ID.

If the determination result is positive (Yes in S207), the interview content registration unit 12 acquires, from the tree structure 123-2 (FIG. 10), a tree structure (route) related to each route to an acquired data item ID related to a leaf node among the acquired data item IDs (that is, an acquired data item ID that is a search starting point in step S206) acquired in step S204 or S205 from an acquired data item ID serving as a search source of the leaf node of which the target acquisition timing is an “acquisition timing”(S208). Note that a plurality of tree structures (routes) may be acquired.

Subsequently, loop processing including steps S209 to S214 is executed for each acquired tree structure (route). A tree structure that is a processing target in the loop processing will be referred to as a “target tree structure”.

In step S209, the interview content registration unit 12 acquires a value of the “reason input flag” of an acquired data item ID of which the “answer acquisition node flag” is 1 in the target tree structure (that is, an acquired data item ID of a leaf node; hereinafter referred to as a “reference source acquired data item ID”) from the acquired data item ID attribute table 123-1 (FIG. 9) (S209). Subsequently, the interview content registration unit 12 determines whether the value of the “reason input flag” is 1 (that is, whether or not the reference source acquired data item ID is a record candidate item related to the intervention) (S210).

If the value of the “reason input flag” is 0 (if the reference source acquired data item ID is a record candidate item related to target person understanding) (No in S210), steps S211 to S214 are not executed for the target tree structure.

If the value of the “reason input flag” is 1 (if the reference source acquired data item ID is a record candidate item related to intervention) (Yes in S210), the interview content registration unit 12 determines whether or not the “reason reference destination Id” of the reference source acquired data item ID is null (S211). As described above, the “reason reference destination Id” is an acquired data item ID that is a reason candidate (for intervention). The value of the “reason input flag” of the reference source acquired data item ID is 1. Therefore, here, the “reason reference destination Id” being null indicates a situation in which the acquired data item ID that can be a reason candidate is not set although the user is expected to input the reason.

If the “reason reference destination Id” is null (Yes in S211), the interview content registration unit 12 acquires the acquired data item ID related to the uppermost “target person understanding” in the tree structure 123-2 (FIG. 10) (that is, the entire tree structure) as the “reference destination acquired data item ID” (S212). Here, the reference destination acquired data item ID is an acquired data item ID that serves as an input destination of a reason for the record input by the user in the subsequent processing with respect to the reference source acquired data item ID. In a case where the “reason reference destination Id” is null, since an acquired data item ID to be the input destination of the reason cannot be narrowed down, an acquired data item ID corresponding to the uppermost “target person understanding” is set as a reference destination acquired data item ID that is a starting point of the input destination of the reason.

On the other hand, if the “reason reference destination Id” is not null (No in S211), the interview content registration unit 12 acquires the acquired data item ID registered in the “reason reference destination Id” as the “reference destination acquired data item ID” (S213).

Subsequent to step S212 or S213, the interview content registration unit 12 acquires, from the tree structure 123-2 (FIG. 10), a tree structure up to all the acquired data item IDs (leaf nodes) of which the “answer acquisition node flag” is 1 and the “acquisition timing” is a target acquisition timing, starting from the reference destination acquired data item ID, and connects the acquired tree structure to the reference source acquired data item IDs of the target tree structure (S214). The acquired tree structure is connected under the target tree structure with the reference source acquired data item ID as a connection point.

When the loop processing is ended for all the tree structures acquired in step S208, the interview content registration unit 12 generates an individual interview record input screen with reference to the tree structure (that is, the tree structure in which step S124 is not executed) in which the “reason input flag” of the reference source acquired data item ID is 0 among the tree structures acquired in step S208 and the tree structure acquired in step S214, and displays the screen (S215).

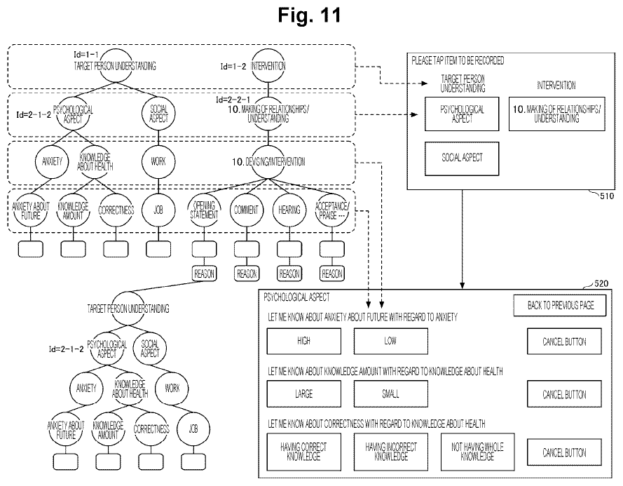

FIG. 11 is a diagram illustrating a first display example of the individual interview record input screen. The tree structure (hereinafter referred to as a “target tree structure”) acquired in step S208 or S214 is illustrated on the left part of FIG. 11. The target tree structure is a tree structure acquired in a case where the “acquired data item ID associated with element” (that is, the acquired data item ID serving as the starting point of the search of the tree structure) acquired in step S204 is “1-1” and “1-2”.

In step S215, first, a main screen 510 configuring the individual interview record input screen is displayed. The main screen 510 has a column for each acquired data item ID (category) corresponding to the uppermost node in the target tree structure. At the top of the column, the acquired data item name (in the example in FIG. 11, “target person understanding” and “intervention” are used) of the corresponding acquired data item ID is displayed as a label. Under the label of each column, a button is displayed for each acquired data item ID (category) corresponding to the child node of the acquired data item ID corresponding to the column. A label of the button is an acquired data item name of a corresponding acquired data item ID.

The user presses a button of a category corresponding to a matter (target person understanding or intervention) that the user has performed in the target interview from among buttons arranged on the main screen 510, and inputs a record regarding the category for each category. Note that, on the main screen 510, a button corresponding to the acquired data item ID including the descendant node of which “acquisition necessity” is “essential” may be highlighted through blinking or the like. This is for preventing the user from forgetting the input to the acquired data item ID since the user is required to input the acquired data item ID of which the “acquisition necessity” is “essential”. Note that, since the record regarding the target person understanding can be a reason candidate for the record regarding the intervention, it is desirable that the record regarding the target person understanding be performed first. Therefore, since the record regarding the target person understanding is ended, the button corresponding to the intervention may be grayed out or the like to be inoperable.

For example, when the user selects a “psychological aspect” button on the main screen 510 in order to input a record regarding the “psychological aspect”, the interview content registration unit 12 displays an input screen 520 illustrated in the lower right part of FIG. 11.

The input screen 520 includes one or more question sentences for guiding the user to input a record and answer options for each question sentence. Each question sentence is generated for each acquired data item ID (hereinafter referred to as a “target terminal acquired data item ID”) related to a leaf node (that is, a record candidate item) among descendant nodes of the acquired data item ID (here, the “psychological aspect”; hereinafter referred to as a “target acquired data item ID”) selected on the main screen 510. According to the target tree structure, there are three leaf nodes which are descendant nodes of the “psychological aspect”. Therefore, the input screen 520 includes three question sentences. For example, the question sentence corresponding to each target terminal acquired data item ID is generated on the basis of the following question sentence generation rule.

“Regarding [acquired data item name of target acquired data item ID], please tell us about [acquired data item name of target terminal acquired data item ID].”

Here, [] indicates replacement with an acquired data item name written in []. For example, regarding one “anxiety about future” of the descendant nodes of the “psychological aspect”, the first [] is replaced with the “sense of anxiety”, and the second [] is replaced with the “anxiety about future”. As a result, a question sentence “Please let me know about the anxiety about the future with regard to anxiety.” is generated.

The answer options are options stored in the acquired data item ID attribute table 123-1 (FIG. 9) with respect to the target terminal acquired data item ID (for example, “anxiety about future”).

For each question sentence, the user selects an option corresponding to understanding of a target person in a target interview or intervening in the target person.

When the input to one input screen 520 is ended, the user returns to the main screen 510, and in a case where there is a button corresponding to an uninput acquired data item ID among the buttons on the main screen 510, the user selects the button and continues the input related to the acquired data item ID (target acquired data item ID).

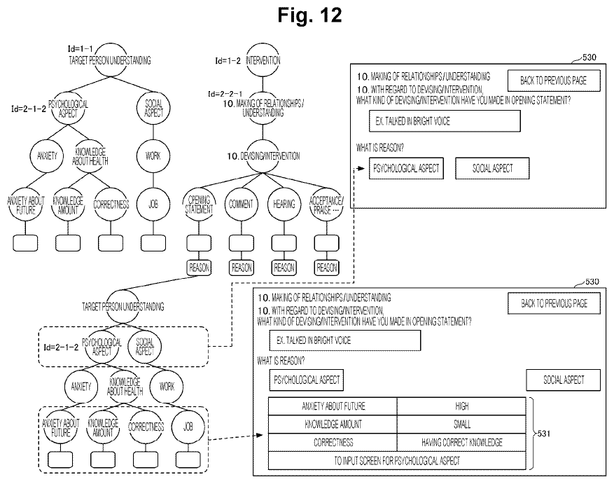

For example, when “10. Making of relationships/understanding” is selected as the target acquired data item ID after inputting the record regarding each category related to the target person understanding, the interview content registration unit 12 displays an input screen 530 as illustrated in the upper right part of FIG. 12.

There are four leaf nodes (hereinafter referred to as “target leaf nodes”) among the descendant nodes of “10. Making of relationships/understanding” which is the target acquired data item ID. However, on the input screen 530 in FIG. 12, for convenience, only one question sentence (“10. With regard to devising/intervention, what kind of devising/intervention have you made in the opening statement?”) related to one target leaf node (“opening statement”) is shown, and question sentences for other target leaf nodes are omitted. In FIG. 12, a format of the question sentence related to the target leaf node is slightly different from the format of the question sentence generation rule described above, but the question sentence generation rule may be changed according to an acquired data item ID. In addition, an answer to the question sentence is in the form of free input. This is because values (options) are not registered in the options 1 to 3 of the acquired data item ID attribute table 123-1 (FIG. 9) with respect to a target terminal acquired data item ID related to the first target leaf node.

The input screen 530 further includes buttons of a “psychological aspect” and a “social aspect” together with a question sentence “What is the reason?”. This is because the “reason input flag” of the target terminal acquired data item ID is 1. Note that a question sentence or the like such as “What is the reason?” is also displayed for question sentences for other target terminal acquired data item IDs (“comment”, “hearing”, “acceptance/praise”, and the like), but is not illustrated in FIG. 12 for convenience.

Here, the reason the interview content registration unit 12 disposes the buttons of “psychological aspect” and “social aspect” on the input screen 530 is that the tree structure (hereinafter referred to as a “reference destination tree structure”) below the reference destination acquired data item ID (“target person understanding” in FIG. 12) is connected to the target leaf node through the operation in step S214 in FIG. 8. That is, in step S214, the reference destination tree structure is connected to the leaf node in order to enable presentation of an option (candidate) of the reason for the leaf node for which the reason needs to be input (the “reason input flag” is 1). In a case where the “reason input flag” of the target terminal acquired data item ID is 1, the interview content registration unit 12 disposes, on the input screen 530, buttons corresponding to nodes (in the example in FIG. 12, “psychological aspect” and “social aspect”) in a hierarchy branched into a plurality of branches in the reference destination tree structure connected to a leaf node related to the target terminal acquired data item ID. Note that, in the tree structure on the left part of FIG. 12, since the leaf node (“Comment”, “Hearing”, “Acceptance/Praise”, and the like) which is a sibling node of the target leaf node (“opening statement”) also has the “reason input flag” of 1, the reference destination tree structure is originally connected, but is not illustrated in FIG. 12 for convenience.

First, the user inputs an answer (for example, “I talked in a bright voice”) to the first question sentence (“10. With regard to devising/intervention, what kind of devising/intervention have you made in the opening statement?”) as a record for the target terminal acquired data item ID.

Subsequently, the user selects a button corresponding to the reason for the answer (for example, “I talked in a bright voice”) from the “psychological aspect” and the “social aspect”. For example, when the reason is related to the “psychological aspect” and the user selects the “psychological aspect” button, the interview content registration unit 12 updates the display content of the input screen 530 as illustrated in the lower right part of FIG. 12.

The lower right input screen 530 in FIG. 12 includes a reason input region 531 for the “psychological aspect”. The reason input region 531 includes, in a table form, the record (answer) input on the input screen 520 (FIG. 11) for each leaf node belonging to the “psychological aspect”. That is, one row corresponds to one leaf node, the first column includes the acquired data item name corresponding to the leaf node, and the second column includes the record (answer) input for the acquired data item ID corresponding to the leaf node. The reason input region 531 presents a reason candidate for the answer input by the user with respect to the first question sentence. That is, each row in the table form corresponds to a reason candidate.

The user inputs a reason by selecting a row corresponding to the reason for executing the intervention indicated by the answer (for example, “I talked in a bright voice”) input to the first question sentence (“10. With regard to devising/intervention, what kind of devising/intervention have you made in the opening statement?”) in the reason input region 531. A plurality of rows may be selected. The interview content registration unit 12 stores the acquired data item name and the answer related to the reason for input for the first target terminal acquired data item ID in association with the target terminal acquired data item ID. For example, when the rows of “anxiety about future” and “high” are selected, the interview content registration unit 12 stores “anxiety about future: high” in association with the first target terminal acquired data item ID. Note that, in this case, it is considered that the user has input a causal relationship that “I talked in a bright voice (intervened)” “since the anxiety for the future (of the target person) is high”.

By the user inputting the reason, it is possible to review why the interviewer executed such an intervention and whether it was an appropriate intervention later. As a result, it is possible to increase the possibility of an effective interview by executing an intervention similar to the intervention devised in the past when an interview with a similar target person (for example, a target person having a small knowledge amount regarding health) is subsequently performed.

When the user finishes inputting the record, the interview content registration unit 12 registers the record content in the individual interview manual recording DB 124 (S216).

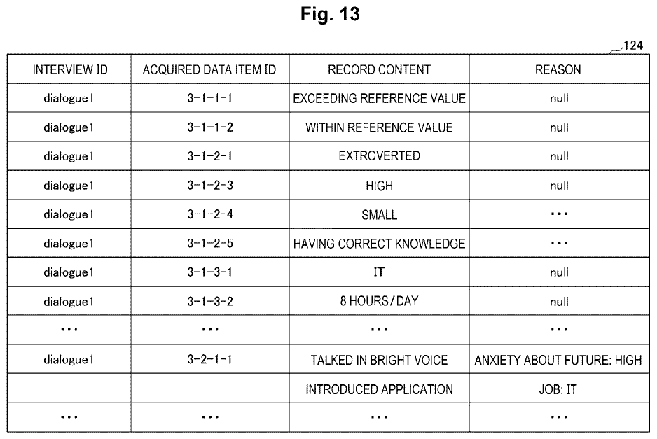

FIG. 13 is a diagram illustrating a configuration example of the individual interview manual recording DB 124. The individual interview manual recording DB 124 is a database in which information indicating how an interviewer understood a target person in each interview and what kind of intervention was executed is recorded. Therefore, the data in the individual interview manual recording DB 124 can be used to review an individual interview. As a result, each interviewer can review what kind of characteristics were included in an interview in which the interviewer was successful, or what kind of characteristics were included in an interview in which seniors or juniors were successful.

In FIG. 13, each record in the individual interview manual recording DB 124 includes items such as an interview ID, an acquired data item ID, record content, and a reason.

The interview ID is an interview ID of a target interview. The acquired data item ID is a target terminal acquired data item ID of which a record (answer) is input on the input screen 520 (FIG. 11) or the input screen 530 (FIG. 12). The record content is the content of the record (answer) input by the user for the acquired data item ID. For example, in a case where an option is selected from among options included in the input screen 520 (FIG. 11), the selected option is registered as the record content. In the case of the free input format as in the first question on the input screen 530 (FIG. 12), the input text is registered as the record content. The reason is a reason that input (selected) by the user with respect to the record content for the acquired data item ID in a case where the “reason input flag” of the acquired data item ID is 1. The reason is expressed in the form of “[acquired data item name]: [input content]”. [Acquired data item name] is an acquired data item name of the acquired data item ID selected as a reason. [Input content] is content of an input to the acquired data item ID selected as a reason. Note that, in the individual interview manual recording DB 124, a set of the acquired data item ID of the record of which a value is registered in the “reason” and the acquired data item ID (hereinafter referred to as a “reason input destination acquired data item ID”) corresponding to the reason ([acquired data item name]: [input content]) (corresponding to the [acquired data item name]) will be hereinafter referred to as “reason record information”. In the example in FIG. 12, a set of “opening statement” and “anxiety about future” corresponds to the reason record information, and the acquired data item ID corresponding to “anxiety about future” corresponds to the reason input destination acquired data item ID. Regarding the target interview, the reason record information exists for each corresponding set. Therefore, there may be a plurality of pieces of reason record information.

Subsequently, the interview content registration unit 12 transmits a reason record information group related to the target interview to the reason input candidate management unit 13 (S217).

### Reason Input Candidate Management Unit 13

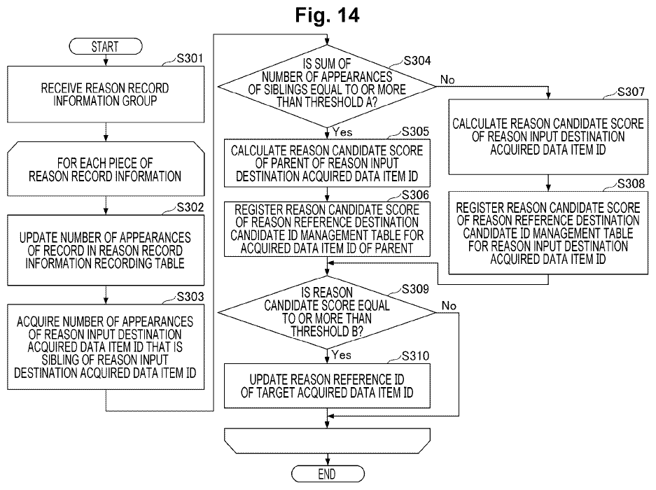

FIG. 14 is a flowchart for describing an example of a processing procedure executed by the reason input candidate management unit 13. The processing procedure in FIG. 14 is executed, for example, in response to the end of the processing procedure in FIG. 8 in order to restrict candidates that are likely to be selected as reasons for intervention and in order to reduce the time and effort required for an interviewer to search for a reason from among a large number of reason candidates when inputting the reason for the intervention. By also using reason record information of a plurality of past interviews, reason candidates that are highly likely to be generally selected for the terminal acquired data item ID related to the intervention are specified. The reason input destination acquired data item ID corresponding to the reason candidate determined to be highly likely to be generally selected for a certain terminal acquired data item ID or the acquired data item ID of the ancestor of the reason input destination acquired data item ID is registered in the “reason reference destination ID” of the terminal acquired data item ID. As a result, for the terminal acquired data item ID, the determination in step S211 in FIG. 8 is positive (Yes), and step S212 is executed. That is, a tree structure under the reason input destination acquired data item ID or the acquired data item ID of the ancestor is connected to the terminal acquired data item ID as a reference destination tree structure. As a result, for example, the reason candidates to be displayed on the input screen 530 in FIG. 12 can be restricted to the candidates having a high possibility of being input. The “reason reference destination ID” in the interview manual recording item DB 123 may be manually registered by, for example, an administrator.

In step S301, the reason input candidate management unit 13 receives a reason record information group transmitted from the interview content registration unit 12. The reason input candidate management unit 13 executes the loop processing in and after step S302 on each piece of reason record information included in the reason record information group. Hereinafter, the reason record information that is a processing target in the loop processing will be referred to as “target reason record information”, and the acquired data item ID and the reason input destination acquired data item ID of the target reason record information will be referred to as a “target acquired data item ID” and a “target reason input destination acquired data item ID”, respectively.

In step S302, the reason input candidate management unit 13 refers to the reason record information recording table 125-1 stored in the reason input candidate management DB 125, and adds 1 to the number of appearances of the reason record information matching the target reason record information (S302).

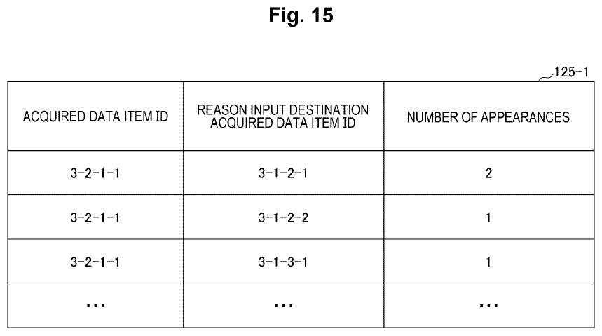

FIG. 15 is a diagram illustrating a configuration example of the reason record information recording table 125-1. As illustrated in FIG. 15, the reason record information recording table 125-1 stores the number of appearances for each piece of reason record information (an acquired data item ID and a reason input destination acquired data item ID). The number of appearances is the number of times the same reason record information has been registered in the individual interview manual recording DB 124 across all the past interviews. That is, the number of appearances is the number of times the record for the acquired data item ID related to the “reason input destination acquired data item ID” has been selected as a reason for executing the intervention input to the “acquired data item ID”.

Subsequently, the reason input candidate management unit 13 acquires the number of appearances of a target acquired data item ID for each acquired data item ID (hereinafter referred to as a “reason input destination acquired data item ID that is a sibling”) corresponding to a sibling of the target reason input destination acquired data item ID from the reason record information recording table 125-1 (FIG. 15) (S303). That is, in the reason record information recording table 125-1 (FIG. 15), the number of appearances of all records in which a value of the “acquired data item ID” matches the target acquired data item ID and a value of the “reason input destination acquired data item ID” matches each acquired data item ID corresponding to a sibling of the target reason input destination acquired data item ID is acquired from the reason record information recording table 125-1 (FIG. 15). Each acquired data item ID corresponding to a sibling of the target reason input destination acquired data item ID is an acquired data item ID corresponding to a sibling node (a node belonging to a common parent node) of the target reason input destination acquired data item ID in the tree structure 123-2 (FIG. 10).

Subsequently, the reason input candidate management unit 13 determines whether or not a sum of the number of appearances (the number of appearances of siblings) acquired in step S303 is equal to or more than a preset threshold A (S304). The sum of the number of appearances of siblings is a sum of the number of appearances of the reason input destination acquired data item IDs that are all the siblings. That is, the “sibling” does not include the target reason input destination acquired data item ID.

If the sum of the number of appearances of the siblings is equal to or more than the threshold A (Yes in S304), the reason input candidate management unit 13 calculates a reason candidate score of the acquired data item ID related to the parent node of the sibling (S305). A reason candidate score of a certain acquired data item ID is an index indicating the degree of appropriateness for the reason data item ID to be the “reason reference destination ID” of the target acquired data item ID. The reason candidate score in this case may be calculated, for example, on the basis of the following formula.

$$Reason candidate score = Σ ( {C}_{i} / n )$$

n: the number of siblings having the number of appearances of 1 or more +1 (where +1 is a portion of the target reason input destination acquired data item ID)
Cᵢ: the number of appearances of siblings having the number of appearances of 1 or more and the number of appearances of target reason record information

That is, the reason candidate score in the above example is an average value of the number of appearances of target reason record information and the number of appearances of a sibling node having the number of appearances of 1 or more.

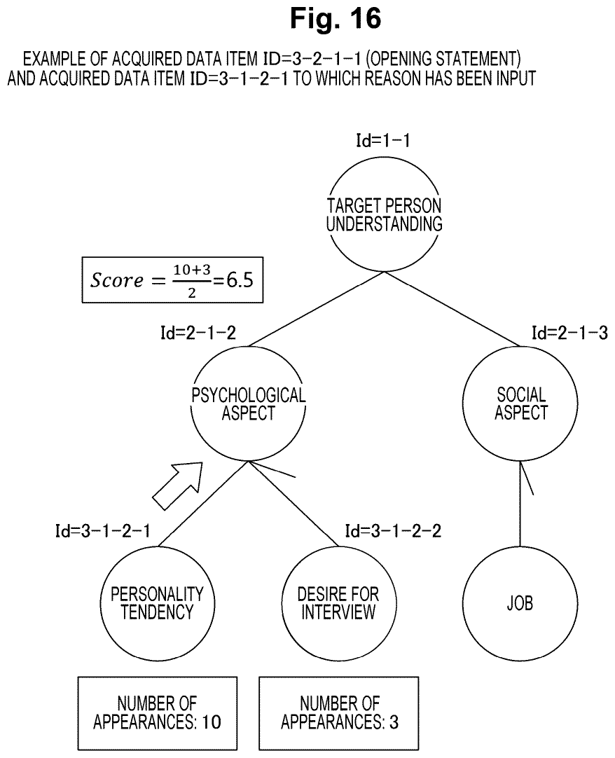

FIG. 16 is a diagram illustrating a calculation example of the reason candidate score of the parent. FIG. 16 illustrates an example of a case where the target acquired data item ID=3-2-1-1 (“opening statement”) and the target reason input destination acquired data item ID=3-1-2-1 (“personality tendency”). In this case, the average of the number of appearances of the target reason input destination acquired data item ID=3-1-2-1 and the number of appearances of the reason input destination acquired data item ID=3-1-2-2 that is a sibling is calculated as a reason candidate score of the acquired data item ID=2-1-2 of the parent (“psychological aspect”).

Subsequently, the reason input candidate management unit 13 updates the reason candidate score for the target acquired data item ID with respect to the acquired data item ID of the parent of the target reason input destination acquired data item ID in the reason reference destination candidate ID management table 125-2 of the reason input candidate management DB 125 (S306).

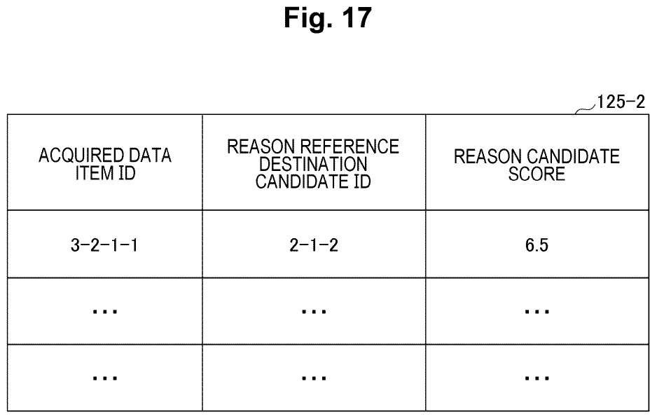

FIG. 17 is a diagram illustrating a configuration example of the reason reference destination candidate ID management table 125-2. As illustrated in FIG. 17, each record of the reason reference destination candidate ID management table 125-2 includes items such as an “acquired data item ID”, a “reason reference destination candidate ID”, and a “reason candidate score”. Each record indicates that the degree of appropriateness of the acquired data item ID related to the “reason reference destination candidate ID” for the “acquired data item ID” being a reason is a “reason candidate score”. Therefore, in step S306, a value of the “reason candidate score” of the record in which the value of the “acquired data item ID” matches the target acquired data item ID and the value of the “reason reference destination candidate ID” matches the acquired data item ID of the parent of the target acquired data item ID is updated to the reason candidate score calculated in step S305. In a case where there is no corresponding record, the record may be newly registered. Note that the first record in FIG. 17 corresponds to the example in FIG. 16.

As described above, in the reason reference destination candidate ID management table 125-2 (FIG. 17), the reason candidate score is registered for the parent of the target reason input destination acquired data item ID. The reason candidate score of the parent is calculated on the basis of the number of appearances of the target reason input destination acquired data item ID and the number of appearances of the reason input destination acquired data item ID that is a sibling. This is based on the idea that in the tree structure 123-2 (FIG. 10) a child takes over the features of a parent. By using the acquired data item ID of the parent as the reason reference destination candidate ID for the target acquired data item, even in a case where only some of the children of the parent are selected as the reason in the past, it is possible to avoid that the reason candidate for the target acquired data item is excessively limited to only the child, and it is possible to present, to the user, other children (that is, the siblings) that are considered to have taken over a common feature with the child as a reason candidate. Note that a reason candidate score may be calculated for any acquired data item ID of the ancestor of the parent instead of the parent according to a method similar to the parent, and the reason candidate score of the acquired data item ID of the ancestor with respect to the target acquired data item ID may be updated in the reason reference destination candidate ID management table 125-2 (FIG. 17).

On the other hand, if the sum of the number of appearances of the siblings is less than the preset threshold A (No in S304), the reason input candidate management unit 13 calculates a reason candidate score for the target reason input destination acquired data item ID (S307). The reason candidate score in this case may be calculated, for example, on the basis of the following formula.
Reason candidate score=C
C: the number of appearances of target reason input destination acquired data item ID

Subsequently, the reason input candidate management unit 13 updates the reason candidate score of the target reason input destination acquired data item ID for the target acquired data item ID in the reason reference destination candidate ID management table 125-2 (FIG. 17) of the reason input candidate management DB 125 (S308). Specifically, a value of the “reason candidate score” of the record in which the value of the “acquired data item ID” matches the target acquired data item ID and the value of the “reason reference destination candidate ID” matches the target reason input destination acquired data item ID is updated to the reason candidate score calculated in step S307. In a case where there is no corresponding record, the record may be newly registered.

Subsequent to step S306 or S308, the reason input candidate management unit 13 determines whether the reason candidate score of the record (hereinafter referred to as an “update record”) in the reason reference destination candidate ID management table 125-2 (FIG. 17) updated in step S306 or S308 is equal to or more than a threshold B (S309).

If the reason candidate score is equal to or more than the threshold B (Yes in S309), the reason input candidate management unit 13 updates the “reason reference destination ID” of the target acquired data item ID in the interview manual recording item DB 123 (FIG. 9) on the basis of the update record (S310). Specifically, the reason input candidate management unit 13 adds the value of the “reason reference destination candidate ID” of the update record to the “reason reference destination ID” of the record in which the value of the “acquired data item ID” matches the “acquired data item ID” (that is, the target acquired data item ID) of the update record in the interview manual recording item DB 123 (FIG. 9).

Values of the thresholds A and B may be changed according to the number of interviews registered in the interview manual recording item DB 123. As the number of interviews increases, the thresholds A and B may be increased.

### Interview Case Visualization Unit 14

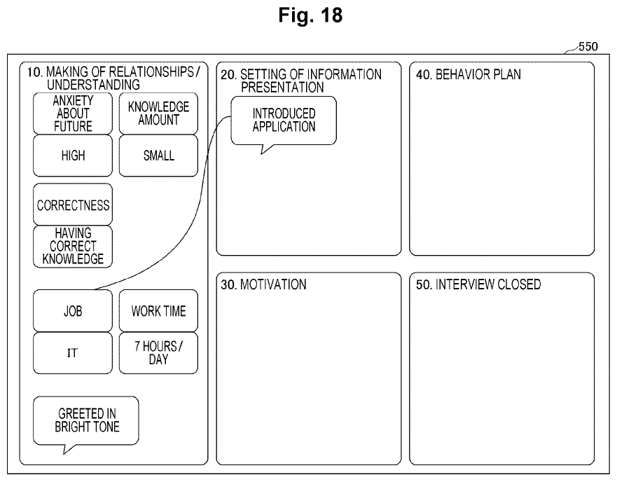

First, a screen displayed by the interview case visualization unit 14 (hereinafter referred to as a “visualization screen 550”) will be described. FIG. 18 is a diagram illustrating a display example of the visualization screen 550. As illustrated in FIG. 18, the visualization screen 550 is divided into a plurality of regions for each conversation process element. Hereinafter, each region after division will be referred to as a “visualization region”.

According to the visualization screen 550, how an interviewer understood a target person and how an intervention was executed are visualized for each individual interview, so that it is possible to support the review of each interview. As a result, the interviewer can review what kind of characteristics were included in an interview in which the interviewer was successful or what kind of characteristics were included in an interview in which seniors or juniors were successful. In order to display such a visualization screen 550, the interview case visualization unit 14 executes the following processing procedure.

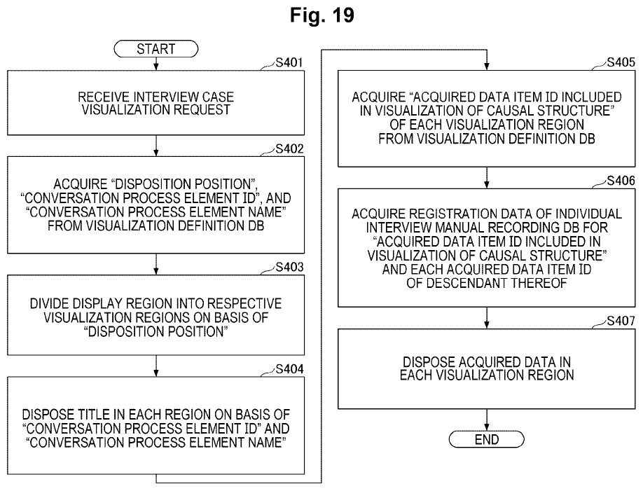

FIG. 19 is a flowchart for describing an example of a processing procedure executed by the interview case visualization unit 14. The processing procedure in FIG. 19 is executed when an interviewer or a colleague reviews a target interview at any timing after the interviewer inputs a record regarding the target interview (after the processing procedure in FIG. 8 is ended). Cases of the target interview are visualized to be useful for learning and discussion in a workplace by reviewing the target interview.

In step S401, the interview case visualization unit 14 accepts a request for visualizing an interview case from the user. In the visualization request, an interview ID (hereinafter referred to as a “target interview ID”) of an interview that is a visualization target (hereinafter referred to as a “target interview”) is designated.

Subsequently, the interview case visualization unit 14 acquires a “disposition position”, a “conversation process element ID”, and a “conversation process element name” for each visualization region from the visualization definition DB 126 (S402).

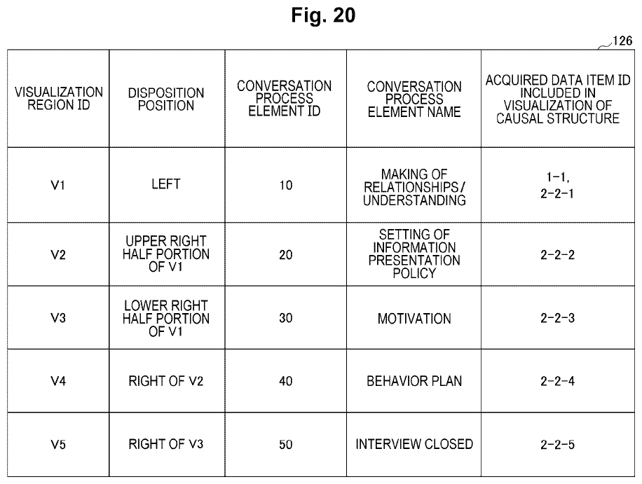

FIG. 20 is a diagram illustrating a configuration example of the visualization definition DB 126. As illustrated in FIG. 20, the visualization definition DB 126 stores in advance a “visualization region ID”, a “disposition position”, a “conversation process element ID”, a “conversation process element name”, an “acquired data item ID to be included in visualization of causal structure”, and the like for each visualization region.

The “visualization region ID” is identification information of the visualization region. The “disposition position” is information indicating a position (range) where the visualization region is disposed in a display region of the visualization screen 550. The “conversation process element ID” and the “conversation process element name” are a conversation process element ID and a conversation process element name corresponding to the visualization region. The “acquired data item ID included in visualization of causal structure” is information indicating an acquired data item ID corresponding to the visualization region. In each visualization region, a figure corresponding to the acquired data item ID of which a record is recorded in the individual interview manual recording DB 124 with regard to the target interview is disposed, and the “acquired data item ID to be included in visualization of causal structure” is information indicating in which visualization region the acquired data item ID is to be disposed.

Subsequently, the interview case visualization unit 14 divides the display region of the visualization screen 550 on the basis of the “disposition position” of each visualization region (S403). In this case, scrolling of the display region may be assumed. That is, a larger display region of the visualization screen 550 may be defined.

Subsequently, the interview case visualization unit 14 generates a title by connecting the “conversation process element ID” and the “conversation process element name” for each visualization region, and disposes the generated title (S404).

Subsequently, the interview case visualization unit 14 acquires the “acquired data item ID to be included in visualization of causal structure” of each visualization region from the visualization definition DB 126 (S405).

Subsequently, the interview case visualization unit 14 refers to the tree structure 123-2 (FIG. 10) to specify the “acquired data item ID to be included in visualization of causal structure” and the acquired data item ID of the descendant thereof, and acquires data (record content) registered in the individual interview manual recording DB 124 (FIG. 13) with regard to the target interview (in association with the interview ID of the target interview) for each specified acquired data item ID (S406). In this case, the interview case visualization unit 14 acquires the acquired data item name of each acquired data item ID from the interview manual recording item DB 123 (FIG. 9).

Subsequently, the interview case visualization unit 14 disposes a figure element indicating the data (the acquired data item name and the record content) acquired for each acquired data item ID in step S406 in the visualization region corresponding to each acquired data item ID (S407). A visualization region corresponding to a certain acquired data item ID is a visualization region corresponding to a record including the certain acquired data item ID in the “acquired data item ID included in visualization of causal structure” in the visualization definition DB 126. In a case where there is no corresponding visualization region, a visualization region corresponding to a record including the acquired data item ID of the ancestor of the certain acquired data item ID in the “acquired data item ID for visualization of causal structure” is a visualization region corresponding to the certain acquired data item ID.

In this case, the interview case visualization unit 14 expresses the acquired data item ID of the descendant node of the “target person understanding” in the tree structure 123-2 (FIG. 10) by a rounded rectangle including the acquired data item name (for example, “anxiety about future”) in the upper part and the “record content” (for example, “high”) in the lower part. In the tree structure 123-2 (FIG. 10), the interview case visualization unit 14 also expresses the “record content” (for example, “an application has been introduced”) in the form of a balloon for the acquired data item ID of the descendant node of the “intervention”. In addition, if the “reason” of the individual interview manual recording DB 124 is not null for the acquired data item ID, the interview case visualization unit 14 connects a figure corresponding to the acquired data item ID and a figure corresponding to the acquired data item ID corresponding to the “reason” with a curve.

Note that the figure elements corresponding to the acquired data item IDs in the respective visualization regions may be arranged in the order of the acquired data item IDs, may be arranged in the order of the Japanese syllabary of the acquired data item names, or may be arranged in the order of the appearance frequency in the interview manual recording item DB 123.

As described above, according to the present embodiment, it is not necessary for a user to input records for all record candidate items, and it is sufficient if the user inputs a record for a record candidate item associated with a conversation process element actually executed in a conversation process in an interview. Therefore, it is possible to efficiently input a record regarding an interview.

A reason for an intervention may be input by selecting a reason from among records input for a record candidate item related to target person understanding. Also in this respect, it is possible to efficiently input a record regarding an interview.

Note that the present embodiment may be applied to learning of an interview between a teacher and a student at an educational site for a teacher, learning of a community participation promotion conversation for elderly people in an area for a care worker or a volunteer, and the like, in addition to an interview related to health guidance.

Note that, in the present embodiment, the executed conversation process management unit 11 is an example of a selection unit. The interview content registration unit 12 is an example of a registration unit.

Although the embodiment of the present invention has been described in detail above, the present invention is not limited to such a specific embodiment, and various modifications and changes can be made within the scope of the concept of the present invention disclosed in the claims.

### REFERENCE SIGNS LIST

10 Interview record input support apparatus
11 Executed conversation process management unit
12 Interview content registration unit
13 Reason input candidate management unit
14 Interview case visualization unit
100 Drive device
101 Recording medium
102 Auxiliary storage device
103 Memory device
104 CPU
105 Interface device
106 Display device
107 Input device
121 Common conversation process model DB
122 Conversation process execution recording DB
123 Interview manual recording item DB
124 Individual interview manual recording DB
125 Reason input candidate management DB
126 Visualization definition DB
B Bus

1. An interview-record-entering support apparatus comprising:
a memory; and
circuitry configured to:
accept a selection of one or more elements performed in an interview, from among a plurality of elements that constitute a dialogue process between an interviewer and an interviewee;
accept a record regarding the interview for an item associated with any of the selected elements, from among a plurality of items that are candidates for registering regarding the interview to review the interview; and
register the record in the memory.

2. The interview-record-entering support apparatus according to claim 1, wherein
each of the plurality of items is associated with any one of a plurality of categories that classify the plurality of items, and
the circuitry is configured to accept the record regarding the interview for a given item, among the plurality of items, belonging to a given category that is associated with any of the selected elements among the plurality of categories.

3. The interview-record-entering support apparatus according to claim 2, wherein
the plurality of categories include a first category related to understanding of the interviewee, and a second category related to an intervention on the interviewee, and
the circuitry is configured to:
accept, for one or more first items among the plurality of items belonging to the first category, the record that is regarding matters that the interviewer has understood about the interviewee in the interview; and
accept, for one or more second items among the plurality of items belonging to the second category, the record that is regarding (i) the intervention performed by the interviewer on the interviewee in the interview and (ii) a reason for the intervention.

4. The interview-record-entering support apparatus according to claim 3, wherein the circuitry is configured to present the record accepted for a given first item of the first items, as a given candidate for the reason for the intervention that is accepted for a given second item of the second items.

5. The interview-record-entering support apparatus according to claim 4, wherein
the circuitry is configured to present, as the given candidate for the reason for the intervention accepted for the given second item, the record accepted for the given first item, and
for the given first item, the number of times the record has been selected as the given candidate for the reason for the intervention accepted for the given second item in the past is equal to or greater than a threshold.

6. The interview-record-entering support apparatus according to claim 4, wherein
the plurality of categories have a parent-child relationship, and
the circuitry is configured to present, as the given candidate for the reason for the intervention accepted for the given second item, the record that is accepted for one or more other first items,
the one or more other first items are descendants of the same category as the given first item, and
a total number of times the record has been selected as the given candidate for the reason for the intervention accepted for the given second item in the past is equal to or greater than a threshold.

7. An interview-record-entering support method executed by a computer, comprising:
accepting a selection of one or more elements performed in an interview, from among a plurality of elements that constitute a dialogue process between an interviewer and an interviewee;
accepting a record regarding the interview for an item associated with any of the selected elements, from among a plurality of items that are candidates for registering regarding the interview to review the interview; and
registering the record in a memory.

8. A non-transitory computer readable storage medium storing a program configured for causing a computer to execute a method, the method comprising:
accepting a selection of one or more elements performed in an interview, from among a plurality of elements that constitute a dialogue process between an interviewer and an interviewee;
accepting a record regarding the interview for an item associated with any of the selected elements, from among a plurality of items that are candidates for registering regarding the interview to review the interview; and
registering the record in a memory.
